(19) United States
(12) Patent Application Publication
(10) Pub. No.: US 2020/0369720 A1
(43) Pub. Date: Nov. 26, 2020

(54) CHARACTERIZATION OF DOMAIN-SPECIFIC CHARGE VARIANTS OF ANTIBODIES

(71) Applicant: Regeneron Pharmaceuticals, Inc., Tarrytown, NY (US)

(72) Inventors: Sophia Liu, White Plains, NY (US); Jennifer NGUYEN, Pleasantville, NY (US); Michael ROSCONI, New City, NY (US); Erica PYLES, New City, NY (US)

(21) Appl. No.: 16/882,453

(22) Filed: May 23, 2020

Related U.S. Application Data
(60) Provisional application No. 62/852,220, filed on May 23, 2019.

(51) Int. Cl. C07K 1/28; C07K 1/18; C07K 7/06

(52) U.S. Cl. CPC C07K 1/28; C07K 1/18; C07K 2317/52; C07K 2317/31; C07K 7/06

(57) ABSTRACT

The present invention provides methods and systems for analyzing the biophysical characteristics of peptides or proteins, such as antibodies, based on enzymatic digestion-assisted imaged capillary electrophoresis (DiCE) to characterize the domain-specific charge variants, preferably within a bispecific antibody. The methods and systems include treating the protein with digestion enzymes to generate components of the protein, reducing or denaturing the components, and separating the components based on their isoelectric points.

### FIELD

The present invention generally pertains to methods and systems for characterizing antibodies including biophysical characterization of domain-specific variants.

### BACKGROUND

The biophysical properties, including domain-specific variants, of therapeutic peptides and proteins can affect their safety, efficacy and shelf-life. For example, the presence of different charge variants may alter the solubility, binding and stability.

Therapeutic peptides or proteins, such as antibodies, may acquire different variants and become heterogeneous due to various post-translation modifications (PTMs), protein degradation, enzymatic modifications and chemical modifications. These alterations to the biophysical properties may occur at almost any point during and after peptides and proteins are produced. Because these alterations to the biophysical characteristics may affect the safety, efficacy and shelf-life of therapeutic peptides and proteins, it is important to identify different variants for particular therapeutic peptides or proteins and their associated safety, efficacy and shelf-life profiles.

It will be appreciated that a need exists for methods and systems to more efficiently and accurately characterize heterogeneity of therapeutic proteins. Further, methods and systems can provide valuable information to identify charge variants that may be associated with particular binding capacities, biological activities, drug safety and shelf life.

### SUMMARY

The introduction of heterogeneity during the manufacturing of therapeutic peptide or proteins, such as antibodies, can be of concern as it may affect safety, efficacy and quality of therapeutic proteins. The present application provides biophysical characterizations of therapeutic peptides or proteins to analyze domain-specific variants of therapeutic peptides or proteins.

Exemplary embodiments disclosed herein satisfy the aforementioned demands by providing methods and systems for identifying or quantifying charge variants of antibodies using digestion-assisted imaged capillary electrophoresis (DiCE) methods, including isoelectric focusing to detect and quantitate the levels of various domain-specific charge variants within antibodies, preferably within bispecific antibodies.

This disclosure provides methods and systems for identifying variants of at least one peptide or protein, comprising: treating the at least one peptide or protein with one or more digestion enzymes to generate components of the at least one peptide or protein; reducing or denaturing the components of the at least one peptide or protein; and subsequently separating two or more components of the at least one peptide or protein.

In some aspects, the digestion enzyme is an immunoglobulin G-degrading enzyme of Streptococcus pyogenes, sialidase, cysteine protease, endopeptidase, papain, endoproteinase Lys-C, pepsin, trypsin, carboxypeptidase B, protease or a combination thereof.

In some aspects, the method for identifying variants of at least one peptide or protein further comprises treating the one or more components with one or more digestion enzymes in multiple phases to generate additional components.

In some aspects, the reducing or denaturing conditions include use of urea, guanidinium chloride, dithiothreitol (DTT), Tris(2-carboxyethyl)phosphine hydrochloride (TCEP), organic solvents, alkaline solution, acid solution or a combination thereof.

In some aspects, the components of the at least one peptide or protein are separated based on charge variants of the components and wherein the physical parameter is charge heterogeneity, molecular weight, charge or combinations thereof.

In some aspects, the components of the at least one peptide or protein are separated using isoelectric focusing electrophoresis method, a capillary isoelectric focusing electrophoresis method, an imaged capillary isoelectric focusing electrophoresis method, a chromatography coupled capillary electrophoresis method, a chromatography coupled imaged capillary electrophoresis method, a cation-exchange chromatography method or a liquid chromatography-mass spectrometry method.

In some aspects, the method for identifying charge variants of at least one peptide or protein further comprises generating a separation profile.

In some aspects, the method for identifying charge variants of at least one peptide or protein further comprises quantifying the separated components of the at least one peptide or protein.

In some aspects, the method for identifying charge variants of at least one peptide or protein further comprises identifying the separated components of the at least one peptide or protein.

In some aspects, the method for identifying charge variants of at least one peptide or protein further comprises identifying the components of the at least one peptide or protein based on a comparison of a separation profile for at least one peptide or protein with a different charge variant.

In yet other aspects, the method for identifying charge variants of at least one peptide or protein further comprises quantifying the level of glycation or C-terminal lysine of the identified components.

In some aspects, in the method for identifying charge variants of at least one peptide or protein, the at least one peptide or protein is a bispecific antibody.

In some aspects, the at least one peptide or protein is a drug, an antibody, a bispecific antibody, a monoclonal antibody, a fusion protein, an antibody-drug conjugate, an antibody fragment or a protein pharmaceutical product.

This disclosure, at least in part, provides a system for identifying charge variants of at least one peptide or protein, comprising: at least one peptide or protein; a first digestion enzyme capable of generating components of the at least one peptide or protein; an environment capable of reducing or denaturing the components of the at least one peptide or protein; and an apparatus capable of separating the components of the at least one peptide or protein that have been digested and reduced or denatured by one or more physical parameters in one or more capillaries.

In some aspects, in the system for identifying charge variants of at least one peptide or protein, the environment further comprises a second digestion enzyme capable of treating the components of the at least one peptide or protein.

In some aspects, in the system for identifying charge variants of at least one peptide or protein, the first or second digestion enzyme is an immunoglobulin G-degrading enzyme of Streptococcus pyogenes, sialidase, cysteine protease, endopeptidase, papain, endoproteinase Lys-C, pepsin, trypsin, carboxypeptidase B or protease.

In yet other aspects, in the system for identifying charge variants of at least one peptide or protein, the environment further comprises a reducing or denaturing agent, wherein the reducing or denaturing agent is urea, guanidinium chloride, reducing agents, dithiothreitol (DTT), Tris(2-carboxyethyl) phosphine hydrochloride (TCEP), organic solvents, alkaline solution, acid solution, or a combination thereof.

In some aspects, in the system for identifying charge variants of at least one peptide or protein, the apparatus separates components based on one or more physical parameters in one or more capillaries, including for example where the physical parameter is charge heterogeneity, molecular weight, charge, or combinations thereof.

In some aspects, the apparatus is an isoelectric focusing apparatus, capillary isoelectric focusing electrophoresis apparatus, an imaged capillary isoelectric focusing electrophoresis apparatus, a chromatograph coupled capillary electrophoresis apparatus, a chromatograph coupled imaged capillary electrophoresis apparatus, a cation-exchange chromatograph apparatus, or a liquid chromatography-mass spectrometry apparatus.

In some aspects, in the system for identifying charge variants of at least one peptide or protein, the at least one peptide or protein is a bispecific antibody.

In some aspects, in the system for identifying charge variants of at least one peptide or protein, the at least one peptide or protein is a drug, an antibody, a bispecific antibody, a monoclonal antibody, a fusion protein, an antibody-drug conjugate, an antibody fragment, or a protein pharmaceutical product.

These, and other, aspects of the invention will be better appreciated and understood when considered in conjunction with the following description and the accompanying drawings. The following description, while indicating various embodiments and numerous specific details thereof, can be given by way of illustration and not of limitation. Many substitutions, modifications, additions, or rearrangements may be made within the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows that MAB4 (Fc/Fc*) is derived by combining a single heavy chain from MAB3 (Fc/Fc) and a single heavy chain from MAB1 (Fc*/Fc*). MAB3 has histidine (H) and tyrosine (Y) residues, and MAB1 has arginine (R) and phenylalanine (F).

FIG. 1B shows that MAB2 is a bispecific antibody targeting both ANTIGENC and ANTIGENA. The glycation of MAB2 occurs at a single residue (ResidueX) in the CDR region of the ANTIGENC arm of the heavy chains of MAB2.

FIG. 2 shows an antibody subjected to an enzymatic digestion to generate antibody fragments, such as using immunoglobulin G-degrading enzyme of Streptococcus pyogenes (IdeS) to generate F(ab′)2 and Fc′ fragments. The antibody fragments can be further subjected to reduced and/or denatured conditions and be analyzed using imaged capillary isoelectric focusing (icIEF) electrophoresis.

FIG. 3 shows that MAB4, MAB3, and MAB1 were analyzed using cation-exchange chromatography (CEX, FIG. 3A), imaged capillary isoelectric-focusing (icIEF, FIG. 3B), or chromatography imaged capillary electrophoresis (chromiCE, FIG. 3C).

FIG. 4A shows the cleavage sites of IdeS.

FIG. 4B shows the results of testing IdeS cleavage efficiency which was analyzed by detecting the presence of the intact antibody molecules and antibody fragments (e.g. F(ab′)2 and Fc′) as indicated under peak area percentages.

FIGS. 5A and 5B show digestion conditions of the IdeS digestion reactions for improving cleavage efficiency, including 4 hours or overnight at 37 degree C., detecting the presence of intact antibody molecules indicated as MAB4 DS in FIG. 5A and indicated as monomer in FIG. 5B.

FIGS. 6A and 6B. show the estimated pI (isoelectric point) of MAB4, MAB3 and MAB1.

FIGS. 7A and 7B show the analysis results of Fc′ fragments, when MAB4, MAB3 or MAB1 were subjected to IdeS digestion and icIEF analysis.

FIG. 8A shows the analysis results of the F(ab′)2 fragments which contained VH domains. F(ab′)2 fragments were purified and separated using icIEF to determine the experimental isoelectric points after IdeS digestion. The experimental isoelectric points of intact antibody molecules were determined using icIEF as shown in FIG. 8B.

FIG. 9 shows the analysis results of MAB4, MAB3 or MAB1 under reduced and/or denatured conditions using DiCE to determine the experimental pI of Fc′, F(ab′)2, ANTIGENB-Fd′, ANTIGENA-Fd′, and LC in comparing to intact antibody molecules.

FIG. 10A shows the analysis results of MAB4 under reduced, denatured conditions using DiCE.

FIG. 10B shows the analysis results of the F(ab′)2 and Fc′ fragments of MAB4 under native conditions using icIEF.

FIGS. 11A and 11B show results obtained using DiCE under reduced/denatured conditions.

FIG. 12 shows triplicate analysis of MAB1, MAB4 and MAB3 under reduced, denatured conditions using DiCE to determine the pI values of corresponding peaks.

FIG. 13 shows triplicate analysis of MAB1, MAB4 and MAB3 under reduced, denatured conditions using DiCE to determine the peak area percentages of corresponding peaks.

FIG. 14 shows the identification of the Fc/Fc* peaks using carboxypeptidase B (CPB) to treat MAB4 Fc′ fragments.

FIG. 15 shows the analysis results of detecting glycation of MAB2 using chromiCE.

FIG. 16 shows the analysis results of detecting glycation of MAB2 and its parental monospecific antibodies under reduced, denatured conditions using DiCE.

FIGS. 17A and 17B show the results of analyzing and enriching glycated species and non-glycated species of MAB2 using CEX and icIEF.

FIGS. 18A and 18B show the analysis results of glycation-enriched samples and enriched non-glycated samples in comparing to MAB2 under reduced, denatured conditions using DiCE with relative abundance of peaks shown in FIG. 18A and the average peak area in percentages corresponding to peak numbers shown in FIG. 18B.

FIG. 19 shows the analysis results of charge variants for samples obtained from two cell lines using CEX and icIEF.

FIG. 20 shows the analysis results of charge variants for samples obtained from two cell lines under reduced, denatured conditions using DiCE.

FIG. 21 shows the analysis results of charge variants for samples obtained from two cell lines under reduced, denatured conditions using DiCE.

FIG. 22 shows the analysis results of charge variants enriched by dual pH-salt gradient cation exchange chromatography using intact icIEF.

FIG. 23 shows the analysis results of charge variants enriched by dual pH-salt gradient cation exchange chromatography using DiCE.

FIG. 24 shows the analysis results of enriched charge variants of MAB5 using DiCE.

FIG. 25 shows the analysis results of combo-Eb using DiCE.

### DETAILED DESCRIPTION

There are concerns about drug efficacy, drug potency and patient safety due to the presence of charge variants of therapeutic proteins, since in some cases the changes of the electric charges of the therapeutic proteins may affect binding capacities, biological activities and shelf life. The methods and systems of the present application can provide valuable information regarding charge heterogeneity of therapeutic proteins which have impacts in clinical pharmacology relevant to pharmacokinetics, efficacy and safety for drug administrations, such as the administration of biologics.

This disclosure provides methods and systems to satisfy the aforementioned demands by providing methods and systems for analyzing the biophysical characteristics of peptides or proteins, such as antibodies. The methods and systems of the present application are based on enzymatic digestion-assisted imaged capillary electrophoresis (DiCE) to characterize the charge variants of the peptide or protein, such as the domain-specific charge variants, preferably in comparing the charge variants between a bispecific antibody and its parental monospecific antibody. The variants can be characterized by biophysical parameters including charge heterogeneity, molecular weight, charge, or combinations thereof. The methods and systems of the present application can be used for all subclasses of human immunoglobulins. The peptides or proteins in the methods or systems of the present application can be, for example, a drug, an antibody, a bispecific antibody, a monoclonal antibody, a fusion protein, an antibody-drug conjugate, an antibody fragment, or a protein pharmaceutical product.

Therapeutic proteins, such as monoclonal antibody products, are extraordinarily heterogeneous due to the presence of various post-translation modification, enzymatic and chemical modifications, such as glycosylation, deglycosylation, amidation, deamidation, oxidation, glycation, terminal cyclization, C-terminal lysine variation, C-terminal arginine variation, N-terminal pyroglutamate variation, C-terminal glycine amidation, C-terminal proline amidation, succinimide formation, sialylation or desialylation. In addition, aggregation, degradation, denaturation, fragmentation, or isomerization of protein products can also introduced charge heterogeneity. Table 1 shows exemplary chemical degradation pathways and their impacts on changing the electric charges of peptides or proteins.

TABLE 1
Chemical degradation pathways of charge variants

| Major chemical | | Species |
|---|---|---|
| degradation pathways | Effect | formed |
| Sialylation | COOH addition | Acidic |
| Deamidation | COOH formation | Acidic |
| C-terminal lysine cleavage | Loss of NH2 | Acidic |
| Adduct formation | COOH formation or loss of NH2 | Acidic |
| Succinimide formation | Loss of COOH | Basic |
| Methionine, cysteine, lysine, | Conformational change | Basic |
| histidine, tryptophan oxidation | | |
| Disulfide-mediated | Conformational change | Basic |
| Asialylation | Loss of COOH | Basic |
| (terminal Galactose) | | |
| C-terminal lysine and | NH2 formation or loss of COOH | Basic |
| glycine admidation | | |

During the manufacture of a therapeutic peptide or protein, such as a monoclonal antibody, charge heterogeneity is potentially introduced as a result of protein degradation and/or the presence of post-translational modifications (PTMs). Characterization of charge variant forms of a protein within the manufactured drug substance is required to fully understand the correlation between properties of the protein, such as potency, and the physical and chemical changes associated with the charge variants.

In particular, for manufacturing monoclonal antibodies, PTMs on different domains of the antibody may result in different biological effects and may potentially impact the stability, safety and potency of the drug product. Therefore, characterization and routine monitoring of domain-specific modifications is important to ensure the quality of therapeutic antibody products. An immunoglobulin G-degrading enzyme of Streptococcus pyogenes (IdeS) has been used to examine the heterogeneity of individual domains in monoclonal antibody products. IdeS can cleave heavy chains of the antibody below the hinge region, producing F(ab′)2 and Fc′ fragments. These antibody fragments can be further reduced to generate three antibody domains, for example, LC (light chain), Fd′ and Fc′/2, for further characterizations, such as analyzed by liquid chromatography/mass spectrometry, capillary isoelectric focusing, glycan mapping, or reversed phase chromatography. (An, Y et al. (2014) A new tool for monoclonal antibody analysis. mAbs 6(4): 879-893).

The present application provides methods and systems based on DiCE including isoelectric focusing to detect and quantitate the levels of various domain-specific charge variants within antibodies, including bispecific antibodies. In some exemplary embodiments, the methods and systems include an enzymatic digestion using IdeS to generate F(ab′)2 and Fc′ fragments, followed by imaged capillary isoelectric-focusing (icIEF) under reduced, denaturing conditions to separate LC, Fd′ and Fc′/2.

The advantages of the present application include providing a highly sensitive method based on DiCE for detecting and quantifying domain-specific PTMs of the different arms of a bispecific antibody. In some exemplary embodiments, DiCE based methods are used to detect and quantify the site-specific glycation in the complementary determining region (CDR) of a bispecific antibody which targets both ANTIGENC and ANTIGENA. Site-specific glycation of ResidueX in the CDR of the ANTIGENC arm of the ANTIGENCxANTIGENA bispecific antibody could not be accurately quantitated using conventional intact charge analysis, such as icIEF and cation-exchange chromatography (CEX). The advantages of the present application include providing a highly sensitive method based on DiCE for detecting domain-specific charge variants of an antibody in assessing the level of unprocessed C-terminal lysine on the Fc domain.

The present application also provides a highly sensitive method or system based on DiCE for the quantitation of PTMs, which is considered comparable to results obtained using peptide mapping. The methods and systems of the present application provide an orthogonal method comparable to peptide mapping for monitoring specific PTMs to ensure product quality of therapeutic peptides and proteins, such as antibodies, fusion proteins, antibody-drug conjugates (ADCs), and antibodies administered concomitantly.

The advantages of the present application further include a DiCE based method or system to analyze a bispecific antibody and its parental monospecific antibody in a single analysis which can provide a more complete view of the domain-specific charge heterogeneity within bispecific antibody molecules. The use of enzymatic digestions in the DiCE based method or system provides the advantages of exposing differences in the intrinsic charge of the resulting antibody fragments by the digesting an intact bispecific antibody. It allows a more complete separation of the resulting antibody fragments or domains by isoelectric focusing.

The method or system of the present application based on DiCE can be applied to bispecific antibodies to distinguish charge variants pertaining to the individual arms of several bispecific antibodies by providing a rapid, medium-throughput method which requires minimal sample of less than about 0.5 mg. The present application also can be used to accurately quantify levels of certain post-translation modification, such as glycation and unprocessed C-terminal lysine. The present application can be applied beyond bispecific antibodies to more fully characterize charge variants of other modalities, such as antibody-drug conjugates (ADCs) and combination products.

The methods and systems of the present application provide an enzymatic digestion reaction to generate components of the peptide or protein. These components are smaller fragments of the peptide or protein. The components of the peptide or protein can be separated subsequently in reduced and/or denatured condition based on electric charges of the components of the peptide or protein to analyze the charge heterogeneity of individual domains within the peptide or protein. The components of the peptide or protein also can be separated based on the differences in isoelectric points among the components, since these components are smaller fragments which have increased differences in isoelectric points among the components.

In some exemplary embodiments, a bispecific antibody or its parental monospecific antibody is subjected to an enzymatic digestion reaction to generate fragments of the antibody, such as F(ab′)2 and Fc′. These antibody fragments can be subsequently separated based on their electric charges using isoelectric focusing. In some exemplary embodiments, these antibody fragments are further subjected to reduced and/or denatured condition to generate specific domains, such as LC, Fd′, and Fc′/2. These antibody domains can be subsequently separated based on electric charges to analyze the charge heterogeneity of individual domains within the antibody. The differences in isoelectric points among the antibody domains enable the separation with measurable differences in isoelectric points, since these domains have smaller molecular weights in comparison to the intact antibody molecule. These antibody domains have increased differences in isoelectric points among the isoelectric points of the antibody domains.

In other exemplary embodiments, the present application provides a method for identifying charge variants of at least one peptide or protein, comprising: treating the at least one peptide or protein with one or more digestion enzymes to generate components of the at least one peptide or protein; reducing or denaturing the components of the at least one peptide or protein; and separating two or more components of the at least one peptide or protein, wherein the components of the at least one peptide or protein are separated based on charge variants of the components, such as isoelectric focusing. In some aspects, the separation profiles of the components are generated and compared to detect or quantify the levels of modifications of the charge variants. They satisfy the long felt needs of characterizing and quantifying the charge heterogeneity of therapeutic proteins which have impact in clinical pharmacology relevant to pharmacokinetics, efficacy and safety for drug administration.

Considering the limitations of existing methods, an effective and sensitive method for identification, detecting or quantifying charge heterogeneity of therapeutic peptides or proteins has been developed using DiCE based methods.

The term “a” should be understood to mean “at least one”; and the terms “about” and “approximately” should be understood to permit standard variation as would be understood by those of ordinary skill in the art; and where ranges are provided, endpoints are included.

As used herein, the terms “include,” “includes,” and “including,” are meant to be non-limiting and are understood to mean “comprise,” “comprises,” and “comprising,” respectively.

In some exemplary embodiments, the disclosure provides a method for identifying variants of at least one peptide or protein, comprising: treating the at least one peptide or protein with one or more digestion enzymes to generate components of the at least one peptide or protein; reducing or denaturing the components of the at least one peptide or protein; and separating two or more components of the at least one peptide or protein based on isoelectric points of the components. In some exemplary embodiments, in the method for identifying charge variants of at least one peptide or protein, the at least one peptide or protein is a drug, an antibody, a bispecific antibody, a monoclonal antibody, a fusion protein, an antibody-drug conjugate, an antibody fragment, or a protein pharmaceutical product.

As used herein, the term “peptide” or “protein” includes any amino acid polymer having covalently linked amide bonds. Proteins comprise one or more amino acid polymer chains, generally known in the art as “peptide” or “polypeptides.” A protein may contain one or multiple polypeptides to form a single functioning biomolecule. In some aspects, the protein can be an antibody, a bispecific antibody, a multispecific antibody, antibody fragment, monoclonal antibody, host-cell protein or combinations thereof.

As used herein, a “protein pharmaceutical product” includes an active ingredient which can be fully or partially biologic in nature. In some exemplary embodiments, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In some other exemplary embodiments, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment may be produced by various means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains that are linked together, for example, by disulfide linkages. An antibody fragment may optionally comprise a multi-molecular complex.

As used herein, the term “antibody-drug conjugate”, or “ADC” can refer to antibody attached to biologically active drug(s) by linker(s) with labile bond(s). An ADC can comprise several molecules of a biologically active drug (or the payload) which can be covalently linked to side chains of amino acid residues of an antibody (Siler Panowski et al., Site-specific antibody drug conjugates for cancer therapy, 6 mAbs 34-45 (2013)). An antibody used for an ADC can be capable of binding with sufficient affinity for selective accumulation and durable retention at a target site. Most ADCs can have Kd values in the nanomolar range. The payload can have potency in the nanomolar/picomolar range and can be capable of reaching intracellular concentrations achievable following distribution of the ADC into target tissue. Finally, the linker that forms the connection between the payload and the antibody can be capable of being sufficiently stable in circulation to take advantage of the pharmacokinetic properties of the antibody moiety (e.g., long half-life) and to allow the payload to remain attached to the antibody as it distributes into tissues, yet also should allow for efficient release of the biologically active drug once the ADC can be taken up into target cells. The linker can include those that are non-cleavable during cellular processing and those that are cleavable once the ADC has reached the target site. With non-cleavable linkers, the biologically active drug released within the call includes the payload and all elements of the linker still attached to an amino acid residue of the antibody, typically a lysine or cysteine residue, following complete proteolytic degradation of the ADC within the lysosome. Cleavable linkers are those whose structure include a site of cleavage between the payload and the amino acid attachment site on the antibody. Cleavage mechanisms can include hydrolysis of acid-labile bonds in acidic intracellular compartments, enzymatic cleavage of amide or ester bonds by an intracellular protease or esterase, and reductive cleavage of disulfide bonds by the reducing environment inside cells.

As used herein, an “antibody” is intended to refer to immunoglobulin molecules consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CHL CH2 and CH3. Each light chain has of a light chain variable region and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL can be composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” is inclusive of, but not limited to, those that are prepared, expressed, created or isolated by recombinant means, such as antibodies isolated from a host cell transfected to express the antibody. An IgG comprises a subset of antibodies.

### Exemplary Embodiment

Embodiments disclosed herein provide compositions, methods, and systems for identification, detecting or quantifying charge heterogeneity of therapeutic peptides or proteins using DiCE based method.

In some exemplary embodiments, the disclosure provides a method for identifying charge variants of at least one peptide or protein, comprising: treating the at least one peptide or protein with one or more digestion enzymes to generate components of the at least one peptide or protein; reducing or denaturing the components of the at least one peptide or protein; and separating two or more components of the at least one peptide or protein based on charge heterogeneity of the charge variants. In some aspects, the method for identifying charge variants of at least one peptide or protein is used to quantify the levels of modifications of the peptide or proteins.

In some exemplary embodiments, the modification of the peptide or proteins includes post-translation modification, enzymatic, chemical modifications, aggregation, degradation, denaturation, fragmentation or isomerization. In some aspects, the modification of the peptide or proteins includes glycosylation, deglycosylation, amidation, deamidation, oxidation, glycation, terminal cyclization, C-terminal lysine variation, C-terminal arginine variation, N-terminal pyroglutamate variation, C-terminal glycine amidation, C-terminal proline amidation, succinimide formation, sialylation, desialylation, adduct formation, disulfide-mediated modification, asialylation (terminal galactose), or C-terminal lysine and glycine admidation. In some aspects, the modification of peptide or protein includes oxidation of methionine, cysteine, lysine, histidine or tryptophan.

In some aspects, in the method for identifying charge variants of at least one peptide or protein, the digestion enzyme is an immunoglobulin G-degrading enzyme of Streptococcus pyogenes, sialidase, cysteine protease, endopeptidase, papain, endoproteinase Lys-C, pepsin, trypsin, carboxypeptidase B, protease, or a combination thereof. In some aspects, the enzymatic digestion was conducted using IdeS by preparing about 100 μl of antibody sample at about 5 mg/mL in 1× DPBS (about 0.5 mg total protein).

In some exemplary embodiments, the components of the peptide or protein are separated based on charge heterogeneity of the charge variants using isoelectric focusing electrophoresis method, a capillary isoelectric focusing electrophoresis method, an imaged capillary isoelectric focusing electrophoresis method, a chromatography coupled capillary electrophoresis method, a chromatography coupled imaged capillary electrophoresis method, a cation-exchange chromatography method, or a liquid chromatography-mass spectrometry method.

In some exemplary embodiments, in the method for identifying charge variants of at least one peptide or protein, reducing or denaturing conditions include use of urea, guanidinium chloride, dithiothreitol (DTT), Tris(2-carboxyethyl)phosphine hydrochloride (TCEP), organic solvents, alkaline solution, acid solution, or a combination thereof. In some aspects, the components of the peptide or protein are reduced and denatured using about 1-10M guanidinium chloride, about 3-8 M guanidinium chloride, or preferably about 6M guanidinium chloride, with about 1-50 mM TCEP, about 5-15 mM TCEP, or preferably with about 10 mM TCEP, incubated at room temperature for about 0.1-2 hr, about 0.5-1.5 hr, or preferably about 1 hr.

The subsequent buffer exchange is performed to remove charged buffer components using the buffer preferably containing about 35 mM phosphate, about pH 6.0, about 8 M urea and about 1 mM TCEP. The components of the peptide or protein are subjected to icIEF under the composition preferably containing about 21 mM phosphate, about pH 6.0, about 8 M urea, about 0.6 mM TCEP, about 0.35% (w/v) methyl cellulose, about 4% (v/v) pH 3-10 pharmalytes.

It is understood that the method or system is not limited to any of the aforesaid peptides, proteins, antibodies, protein pharmaceutical products, digestion enzymes, reducing/denaturing conditions, isoelectric focusing electrophoresis, imaged capillary isoelectric focusing electrophoresis, chromatography coupled capillary electrophoresis, chromatography coupled imaged capillary electrophoresis method, cation-exchange chromatography method or a liquid chromatography-mass spectrometry method.

The consecutive labeling of method steps as provided herein with numbers and/or letters is not meant to limit the method or any embodiments thereof to the particular indicated order.

Various publications, including patents, patent applications, published patent applications, accession numbers, technical articles and scholarly articles are cited throughout the specification. Each of these cited references is herein incorporated by reference in its entirety.

The disclosure will be more fully understood by reference to the following Examples, which are provided to describe the disclosure in greater detail. They are intended to illustrate and should not be construed as limiting the scope of the disclosure.

### EXAMPLES

Materials and Reagents Preparation

### 1.1. Monospecific and Bispecific Antibodies.

Several therapeutic antibodies were subjected to biophysical characterizations including bispecific antibodies and their parental monospecific antibodies. The format of a bispecific antibody includes pairing two different heavy chains (HC) with two common light chains (LC), which enables two unique antigen-binding sites targeting two different antigens. For example, MAB4 is a bispecific antibody targeting both ANTIGENB and ANTIGENA. The ANTIGENA arm in heavy chains of MAB4 has a two amino acid substitution. Substitutions of two amino acids in the Fc region of one of the heavy chains of MAB4 abrogate protein A binding, for example, substituted HY with RF referred as star-substitution or Fc*. This star-substitution contributes to the difference in the theoretical isoelectric points between unsubstituted and substituted heavy chains, which may facilitate the antibody purification or separation based on the empirical (e.g., observed or experimental) isoelectric points.

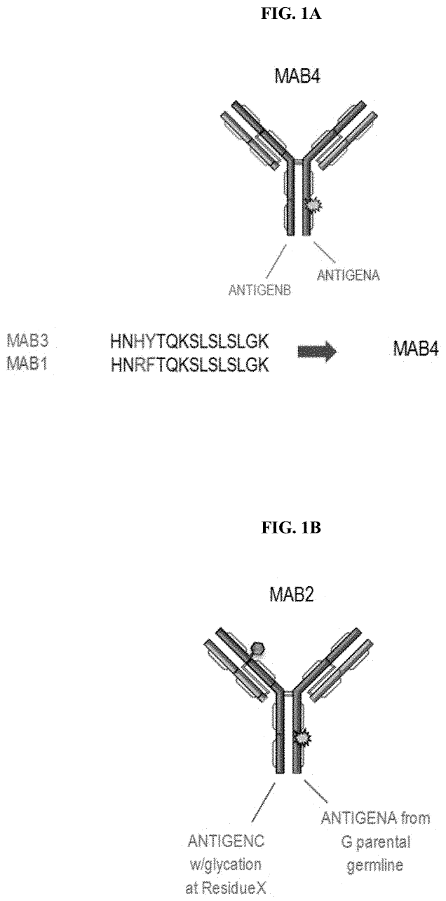

MAB3 is a monospecific antibody targeting ANTIGENB, which does not have star-substitutions in heavy chains, for example, Fc/Fc. MAB1 is a monospecific antibody targeting ANTIGENA, which has star-substitutions in both heavy chains, for example, Fc*/Fc*. MAB4 is a bispecific antibody targeting both ANTIGENB and ANTIGENA. MAB4 (Fc/Fc*) is derived by combining a single heavy chain from MAB3 (Fc/Fc) and a single heavy chain from MAB1 (Fc*/Fc*) as shown in FIG. 1A. In comparing the amino acid sequences between MAB3 and MAB1, they differ in the Fc region of the heavy chain as shown in FIG. 1A. In the corresponding positions as indicated in FIG. 1A, MAB3 has histidine (H) and tyrosine (Y) residues, and MAB1 has arginine (R) and phenylalanine (F).

MAB2 has about 40% glycation and is a bispecific antibody targeting both ANTIGENC and ANTIGENA. The glycation of MAB2 occurs at a single residue ResidueX in the CDR region of the ANTIGENC arm of the heavy chains of MAB2 as shown in FIG. 1B. The glycation at ResidueX has impacts on drug activity and potency. The ANTIGENA arm of the heavy chains of MAB2 is derived from G parental germline, which has a star-substitution in the Fc region.

2.1 Enzymatic Digestion of Antibodies

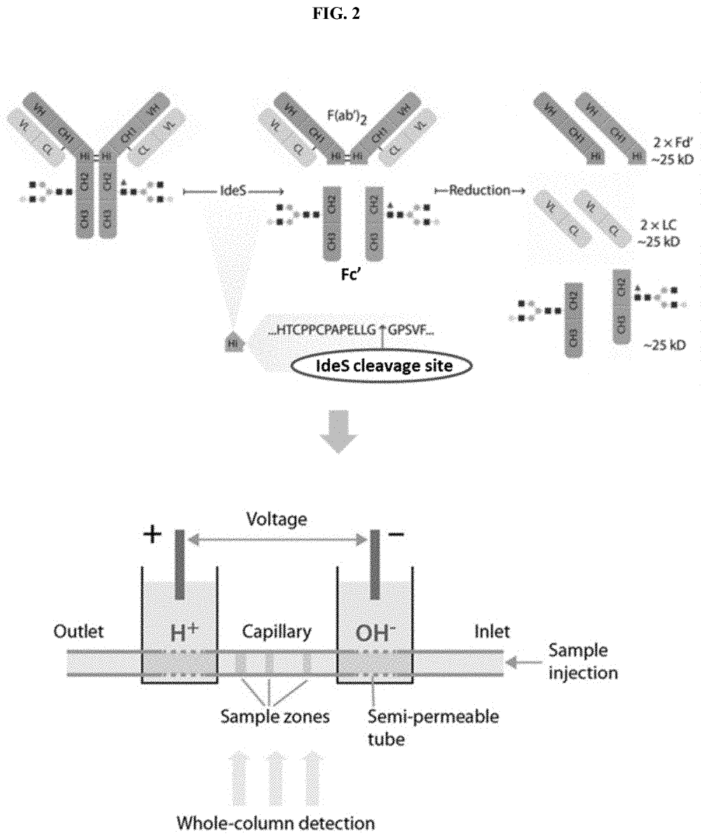

Antibodies are subjected to an enzymatic digestion to generate antibody fragments, such as using immunoglobulin G-degrading enzyme of Streptococcus pyogenes (IdeS) to generate F(ab′)2 and Fc′ fragments as shown in FIG. 2. The F(ab′)2 and Fc′ fragments can be further subjected to reduced and/or denatured conditions to generate antibody domains, such as LC, Fd′ and Fc′/2. For example, when MAB4 (ANTIGENBxANTIGENA, Fc/Fc*) is subjected to IdeS digestion, it generates F(ab′)2 and Fc′ fragments of MAB4. When the F(ab′)2 fragment of MAB4 is subjected to reduced and/or denatured conditions, it generates two Fd′, for example, ANTIGENB-Fd′ and ANTIGENA-Fd′, and two light chains (LC). When the Fc′ fragment of MAB4 is subjected to reduced and/or denatured conditions, it generates two antibody domains, for example, ANTIGENB arm of Fc′ and ANTIGENA arm of Fc′ (with star-substitution).

Instruments for Isoelectric Focusing.

### 1.1. Workflow of Digestion-Assisted Imaged Capillary Electrophoresis (DiCE)

The method of identifying an electrical charge of a peptide or protein using DiCS includes treating the peptide or protein with an enzymatic digestion reaction to generate components of the peptide or protein, and separating the components of the peptide or protein based on electric charges or isoelectric points of the components. FIG. 2 shows an embodiment of a general DiCE workflow using icIEF electrophoresis to separate the fragments of a bispecific antibody, wherein the bispecific antibody is fragmented using IdeS.

In some exemplary embodiments, the enzymatic digestion was conducted using IdeS by preparing about 100 μl of antibody sample at about 5 mg/mL in 1× DPBS (about 0.5 mg total protein). The sample was subsequently reduced and denatured using about 6M guanidinium chloride with about 10 mM TCEP incubated at room temperature for about 1 hr. The buffer exchange was performed to remove charged buffer components using the buffer containing about 35 mM phosphate, about pH 6.0, about 8 M urea and about 1 mM TCEP. The sample was subjected to icIEF under the composition containing about 21 mM phosphate, about pH 6.0, about 8 M urea, about 0.6 mM TCEP, about 0.35% (w/v) methyl cellulose, about 4% (v/v) pH 3-10 pharmalytes. The undigested material was estimated to be less than about 2% which would not affect the peak quantitation using DiCE.

### Example 1. Analyze Charge Heterogeneities of Antibodies Using CEX, icIEF and chromiCE

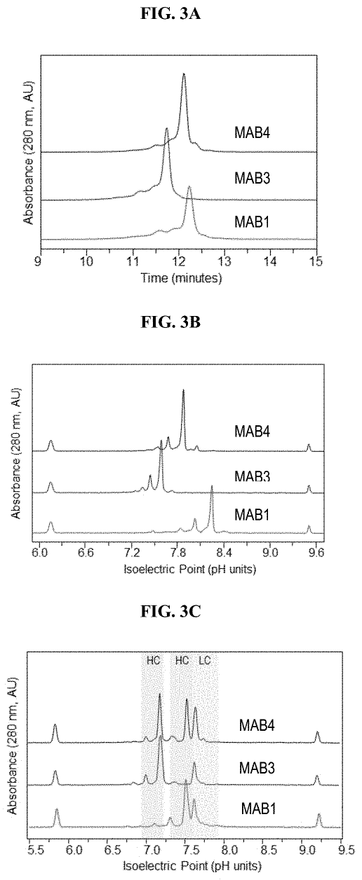

CEX, icIEF or chromatography imaged capillary electrophoresis (chromiCE) was used to analyze the charge heterogeneities of antibodies. Therapeutic antibodies, e.g. MAB4, MAB3, and MAB1, were used for the analysis as shown in FIG. 3.

CEX was used to analyze the surface charge of the intact antibody molecule, which provided low-to-medium resolution as shown in FIG. 3A. icIEF was used to analyze the overall, for example, intrinsic, charge of the intact antibody molecule, which provided high resolution with baseline separation based on the isoeletric point of the intact molecule as shown in FIG. 3B. chromiCE was used to analyze the intrinsic charges of individual heavy chains (HC) and light chains (LC) of the antibody, wherein the antibodies were analyzed under reduced and/or denatured conditions. HC and LC were separated using a size exclusion chromatography (SEC) method under reduced and/or denatured conditions, such as in the presence of about 6 M guanidinium chloride and Tris(2-carboxyethyl)phosphine hydrochloride (TCEP) with about 1 hr incubation, followed by the analysis of icIEF. When there was significant overlap between the isoelectric points of the HC and LC, the HC and LC were not able to be separated completely which resulted overlapped HC and LC peaks as shown in FIG. 3C. The analysis results of CEX, icIEF and chromiCE, for example, the conventional charge-based assays, were unable to differentiate the charge differences between two unique heavy chains within the bispecific antibody.

When the analysis was conducted using intact icIEF under reduced and/or denatured conditions, distinct peaks represented the individual HCs were detected and resolved. However, the charge differences between the F(ab′)2 and Fc′ fragments were not differentiable, since the signals were convolved into a single HC signal. These charge variants include charge differences relevant to post-translation modification due to sequence differences in VH domain or constant region (for example, star-substitution).

### Example 2. IdeS Digestion of Antibodies

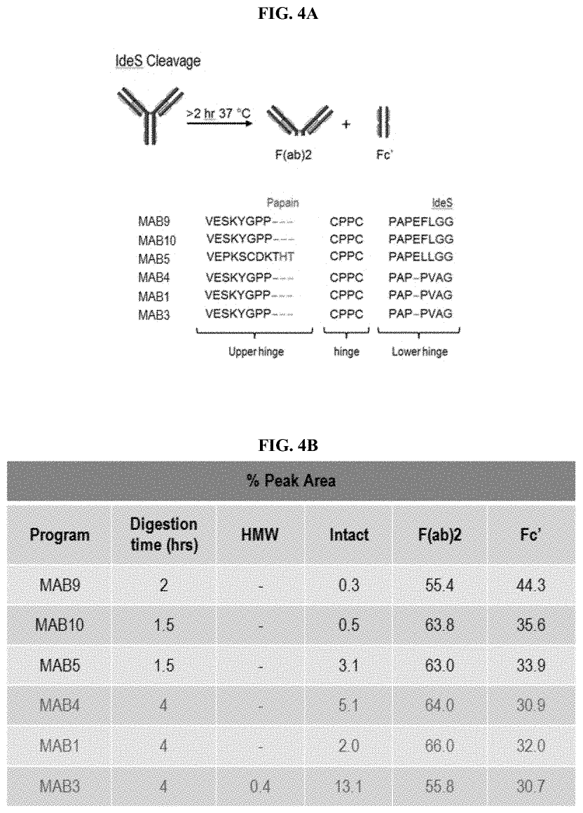

IdeS was used to conduct an enzymatic digestion reaction of antibodies at about 37 degree C. for at least about 2 hr. The IdeS digestion under this condition was efficient in cleaving the immunoglobulin (IgG) molecules to generate antibody fragments, for example, F(ab′)2 and Fc′, as shown in FIG. 4A. Several therapeutic antibodies, for example, MAB9, MAB10, MAB5, MAB4, MAB1 and MAB3, were used for the IdeS digestion and were subsequently subjected to icIEF analysis. The digestion efficiency of IdeS was analyzed by detecting the presence of the intact antibody molecules and antibody fragments (e.g., F(ab′)2 and Fc′) as indicated under peak area percentages as shown in FIG. 4B with the recorded digestion time and monitoring the presence of high molecule weight (HMW) molecules. The GG lower hinge sequence yielded better cleavage efficiency in comparing to the AG lower hinge sequence.

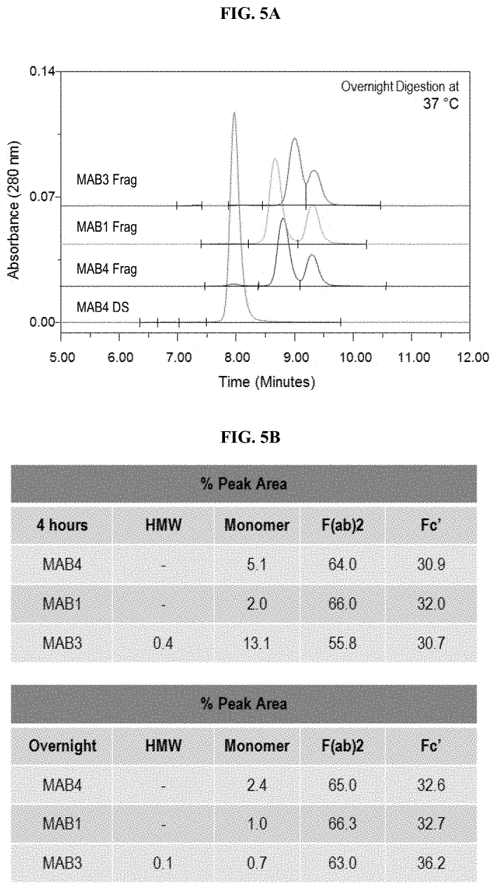

Various digestion conditions of the IdeS digestion reactions were tested to improve cleavage efficiency, such as about 4 hr or overnight at about 37 degree C. The digestion efficiency of IdeS was analyzed by detecting the presence of the intact antibody molecules (indicated as monomer in FIG. 5B) and antibody fragments (e.g., F(ab′)2 and Fc′) as indicated under peak area percentages at retention time as shown in FIG. 5, including monitoring the presence of high molecule weight (HMW) molecules. The undigested MAB4 antibody was used as control (indicated as MAB4 DS in FIG. 5A). The tested results indicate that the cleavage efficiency was molecule-dependent.

### Example 3. Estimated Isoelectric Points in F(ab′)2 and Fc′ Fragments

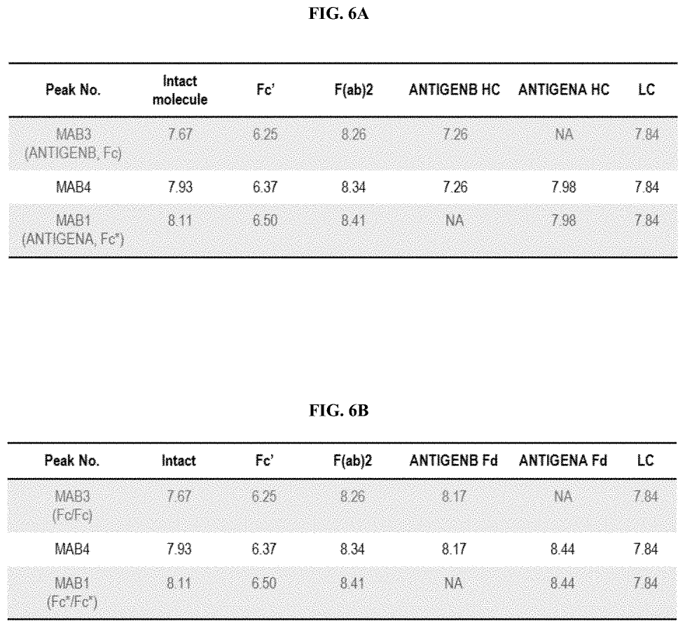

MAB4 (Fc/Fc*) is a bispecific antibody targeting both ANTIGENB and ANTIGENA, which is derived by combining a single heavy chain from MAB3 (ANTIGENB, Fc/Fc) and a single heavy chain from MAB1 (ANTIGENA, Fc*/Fc*) as shown in FIG. 1A. The heavy chains of MAB3 and MAB1 differ in the Fc region. Due to the differences in amino acid sequences, for example, amino acid substitutions from HY to RF, the ANTIGENA heavy chain has a star-substitution in Fc region. The estimated pI (isoelectric point) of MAB4 suggested that the ANTIGENB arm is more acidic than the bispecific antibody, while the ANTIGENA arm is more basic as shown in FIGS. 6A and 6B.

### Example 4. Analysis of F(ab′)2 and Fc′ Fragments

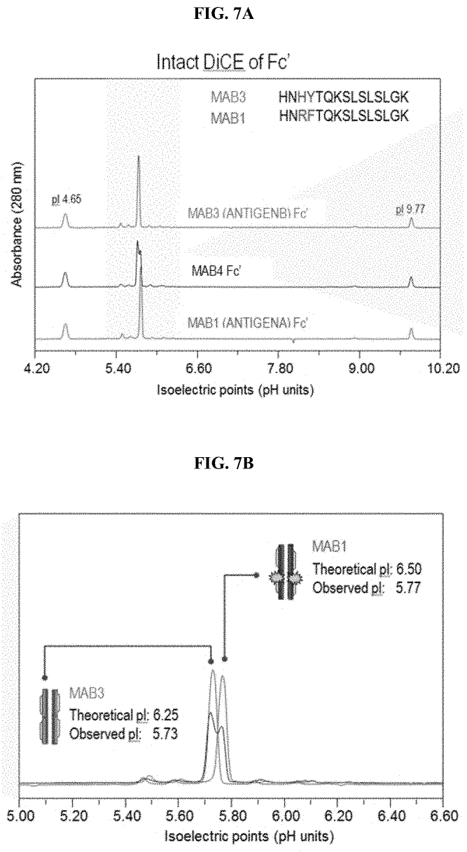

MAB4, MAB3 or MAB1 was subjected to enzymatic digestion including treating the antibodies with IdeS to generate antibody fragments, for example, F(ab′)2 and Fc′ fragments. Subsequently, the antibody fragments were separated based on their electric charges or isoelectric points. For the DiCE workflow, protein concentration at about 0.3 mg was used. For the isoelectric focusing, the ratio of pH 3-10 pharmalytes and pH 8-10.5 pharmalytes was about 3:1. As indicated in FIG. 7A and FIG. 7B, based on the testing results, the Fc′ fragment of MAB1 was more basic in comparing to that of MAB4 and MAB3. The Fc′ fragment of MAB3 was more acidic. The results indicated that the Fc′ fragment of MAB1 was more basic due to the presence of two star-substitutions, which was consistent with the pI estimates. The results also indicate that the Fc′ fragment of MAB4 was more basic in comparing to MAB3 due to the presences of one star-substitution in MAB4. In conclusion, the results indicated that the presence of one or two star-substitutions can make a contribution to a measurable change in isoelectric points for the detection of domain-specific charge variants within a bispecific antibody. The Fc′ fragment with star-substitution was experimentally verified to possess a more basic overall charge than the native Fc′ fragment, which was consistent with the pI predictions.

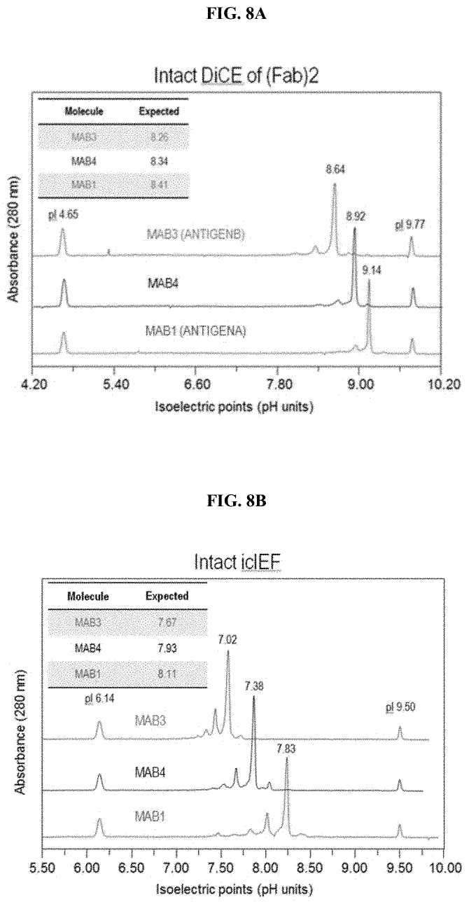

After IdeS digestion, the F(ab′)2 fragments which contained VH domains were purified and separated using icIEF to determine the experimental isoelectric points as shown in FIG. 8A. The experimental isoelectric points of intact antibody molecules were determined using icIEF as shown in FIG. 8B. The results indicated that the differences in experimental isoelectric points among the F(ab′)2 fragments of MAB3, MAB4 and MAB1 were well correlated with the differences among corresponding intact antibody molecules. However, the separation of the F(ab′)2 fragments using icIEF did not provide sufficient differentiation to distinguish the charge difference between two heavy chains, for example, with or without star-substitution, when Fc′ fragments were removed after IdeS digestion.

### Example 5. Analyze Antibody Fragments Under Reduced and/or Denatured Conditions

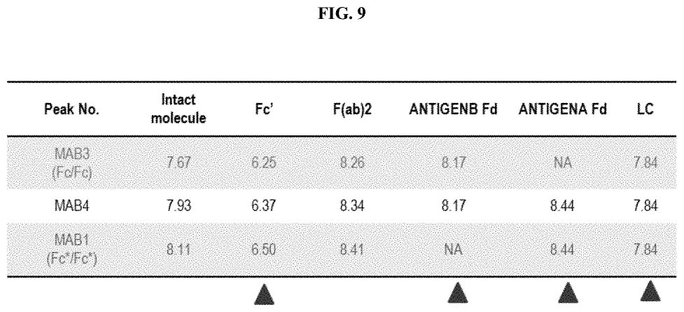

MAB3, MAB4 or MAB1 was digested using IdeS at about 37 degree C. overnight (about 16 hr) to generate antibody fragments, for example, F(ab′)2 and Fc′. The antibody fragments were subsequently treated with about 6M guanidinium chloride and Tris(2-carboxyethyl)phosphine hydrochloride (TCEP) for 1 hr to obtain four or five distinct antibody domains, for example, ANTIGENB-Fd′, ANTIGENA-Fd′, LC (light chain), ANTIGENB arm of Fc′, and/or ANTIGENA arm of Fc′. These antibody fragments and domains were analyzed using icIEF to determine their experimental isoelectric points as shown in FIG. 9.

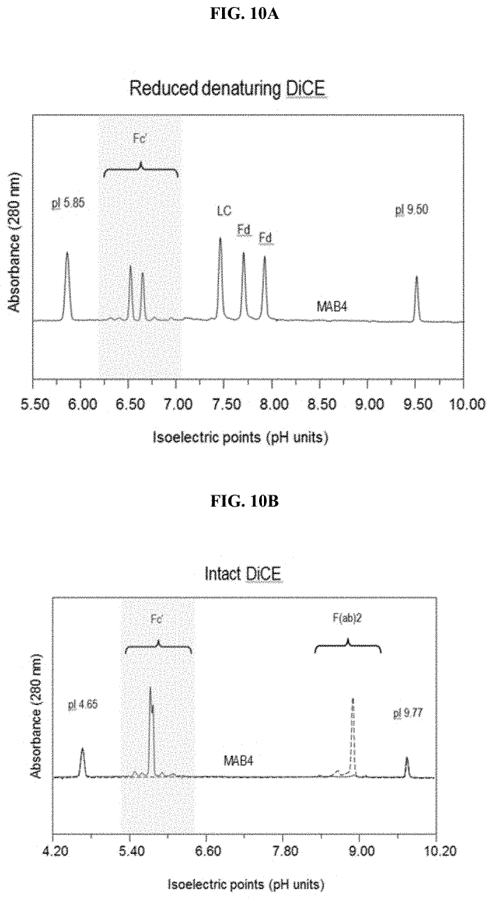

When MAB4 was digested with IdeS and analyzed under reduced and/or denatured condition using icIEF, the results showed 5 distinct peaks corresponding to Fc′, LC and two Fd, with differentiable isoelectric points as shown in FIG. 10A. FIG. 10 B showed the analysis results of F(ab′)2 and Fc′ fragments, when MAB4 was digested with IdeS and analyzed using icIEF without the use of reduced and/or denatured condition. In comparing the results in FIG. 10A and FIG. 10B, the reduced and/or denatured condition accentuated the differences in the isoelectric points for the identification of the domain-specific charge variants of antibodies. In particular, a trio of well-separated peaks corresponding to LC and two distinct Fd′ can be observed as shown in FIG. 10A.

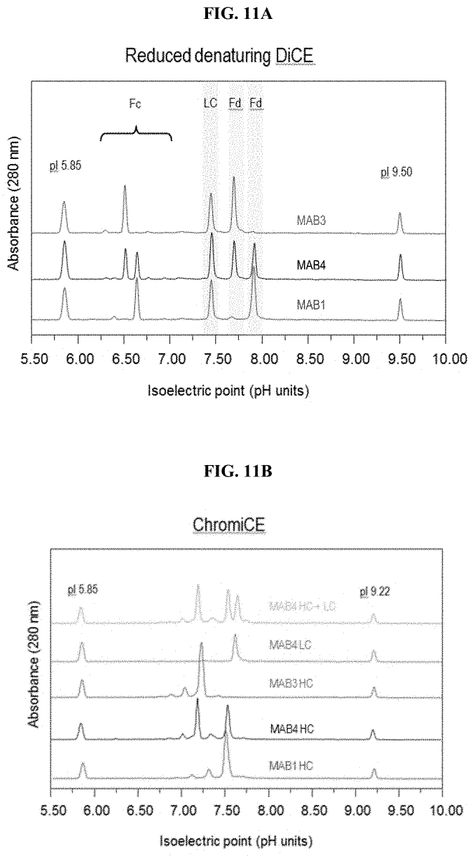

When MAB3 or MAB1 was digested with IdeS and analyzed under reduced and/or denatured conditions using icIEF, the results showed 3 distinct peaks for each antibody with differentiable isoelectric points corresponding to Fc′, LC and Fd′, as shown in FIG. 11A. In comparing the peaks among the results of MAB3, MAB4 and MAB1, the trio of peaks in the results of MAB4 can be identified as common LC, ANTIGENB-Fd′, and ANTIGENA-Fd′ from acidic to basic p1. By simultaneously performing DiCE on the parental monospecific monoclonal antibody and purified Fc′ and F(ab′)2 fragments, the identities of each peak were confirmed. The experimental pI values of the Fd′ fragments were correlated well with their expected pI values regarding MAB4 and its parental monospecific antibodies. Some overlaps in the signals between Fd′ fragments were observed. Some LC signals overlapped with that of the acidic Fd′. Overall, near-baseline separation of three major peaks for each fragment (or domain) was observed.

The results indicated that the pH differences in Fd′ were about 0.3-0.4 pH units obtained using DiCE under reduced/denatured conditions, which was similar to the test results obtained using chromiCE. The pI ranges among HCs obtained using chromiCE was about pH 6.8-7.6 which was about 0.8 pH unit in difference. The pI ranges among Fd′ fragments obtained using DiCE was about pH 7.5-8.0 which was about 0.5 pH units in difference.

As shown in FIGS. 11A and 11B, the results obtained using DiCE under reduced/denatured conditions were complementary to chromiCE. However, when the antibodies were analyzed using DiCE under reduced/denatured conditions, the results were able to show the pI differences between two heavy chains of the bispecific antibody, in particular showing the pI differences among Fc′ and Fd′.

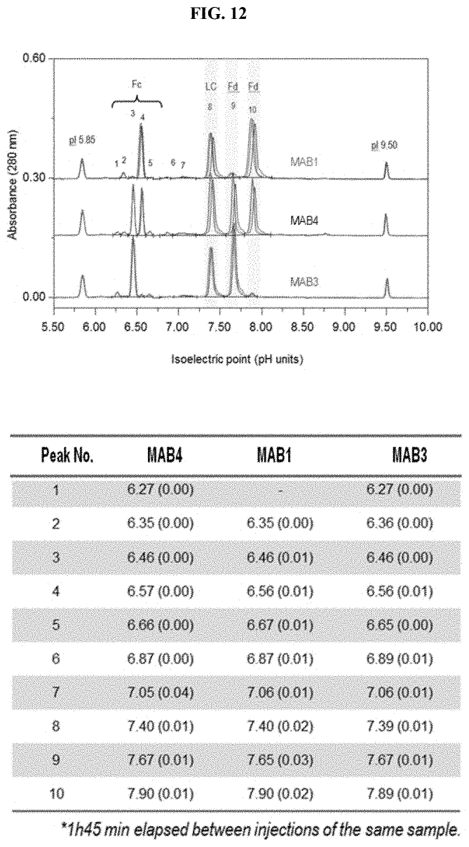

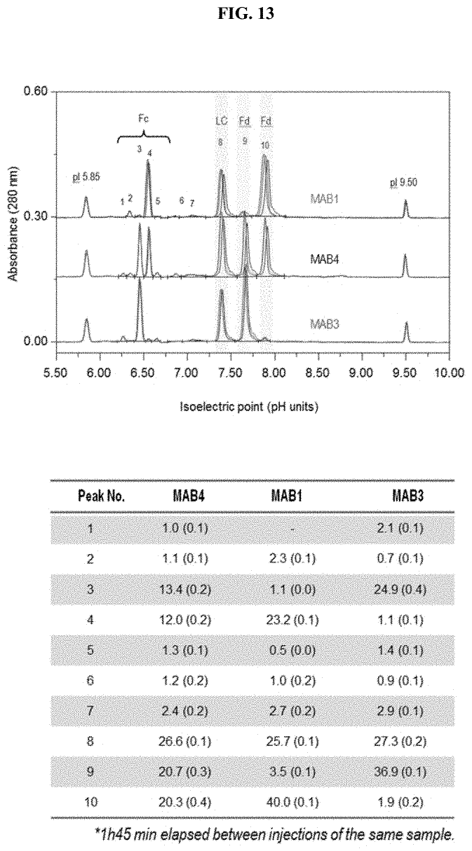

In order to examine the reproducibility of the testing results using DiCE under reduced/denatured conditions, the experiments were duplicated in triplicate injections for MAB1, MAB4 and MAB3 as shown in FIG. 12 and FIG. 13. The pI values of the corresponding peaks were highly reproducible as shown in FIG. 12. The peak area percentages of the corresponding peaks were highly reproducible as well as shown in FIG. 13.

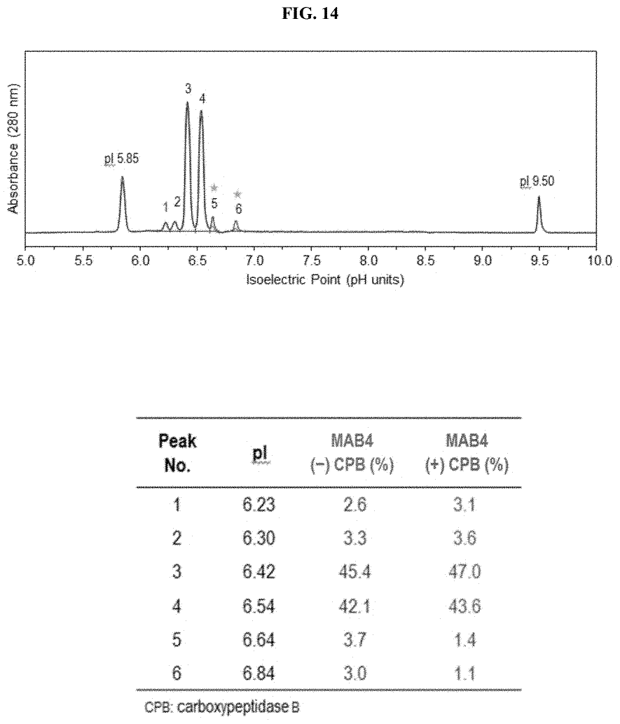

In order to further identify the Fc/Fc* peaks, purified MAB4 Fc′ fragments were treated with carboxypeptidase B (CPB) to specifically cleave the C-terminal lysine. The reduced abundances of peaks 5 and 6 demonstrated that these peaks contained unprocessed C-terminal lysine as shown in FIG. 14.

### Example 6. Identification of Glycation in Bispecific Antibody

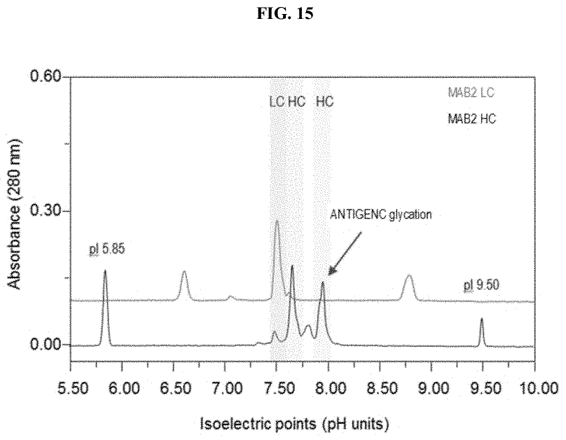

MAB2 is a bispecific antibody targeting both ANTIGENC and ANTIGENA. The glycation of MAB2 occurs at a single residue ResidueX in the CDR region of the ANTIGENC arm of the heavy chains of MAB2. The glycation of MAB2 was unable to be detected using icIEF analysis of the intact antibody molecule. Even though the use of CEX was able to detect glycation of MAB2, the test results obtained using CEX were unable to quantify the levels of glycation due to poor resolution between the peaks corresponding to the glycated species and neighboring peaks. The glycation of the ANTIGENC arm of the heavy chain was slightly visible in chromiCE and overlapped with the peak of heavy chain (at the shoulder of ANTIGENC heavy chain) as shown in FIG. 15. The glycation levels of MAB2 were not able to be accurately quantified using chromiCE due to poor resolution.

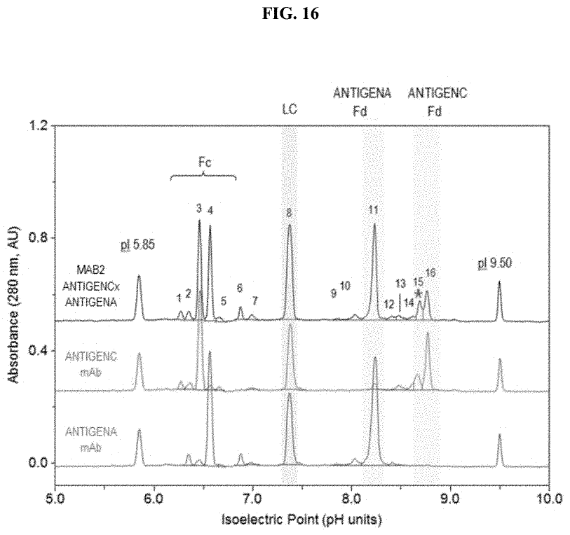

MAB2 and its parental monospecific antibodies, for example, ANTIGENC and ANTIGENA monoclonal antibodies, were subjected to IdeS digestion and analyzed using DiCE under reduced/denatured conditions. The test results of three antibodies were compared side by side for peak identifications as shown in FIG. 16. Peaks 1-6 were identified as corresponding to the Fc domain. Peak 8 was present in all samples and was identified as corresponding to LC. Peak 11 was identified as representing the predominant anti-ANTIGENA Fd′ fragment. Peaks 9-10 could represent the acidic forms of the anti-ANTIGENA Fd′. The anti-ANTIGENC Fd′ fragment appeared as a highly resolved, split peak, for example, peaks 15 and 16, which could represent glycated and non-glycated forms of ANTIGENC. Peaks 12-14 could represent the acidic forms of the anti-ANTIGENC Fd′ fragment.

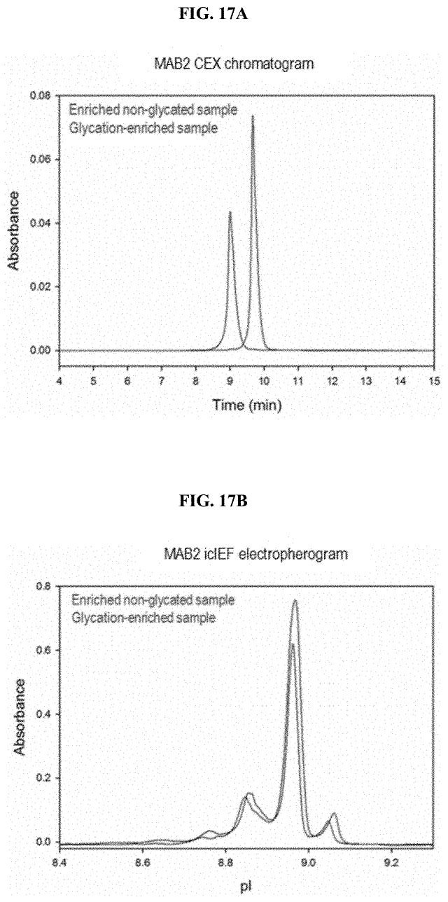

In order to confirm the identities of peak 15 and 16 in corresponding to ANTIGENC, glycated species and non-glycated species were enriched to homogeneity using CEX. The enriched glycated species and non-glycated species have distinct and differentiable profiles in CEX as shown in FIG. 17A. However, the peak profiles were not differentiable when they were analyzed using icIEF as shown in FIG. 17B.

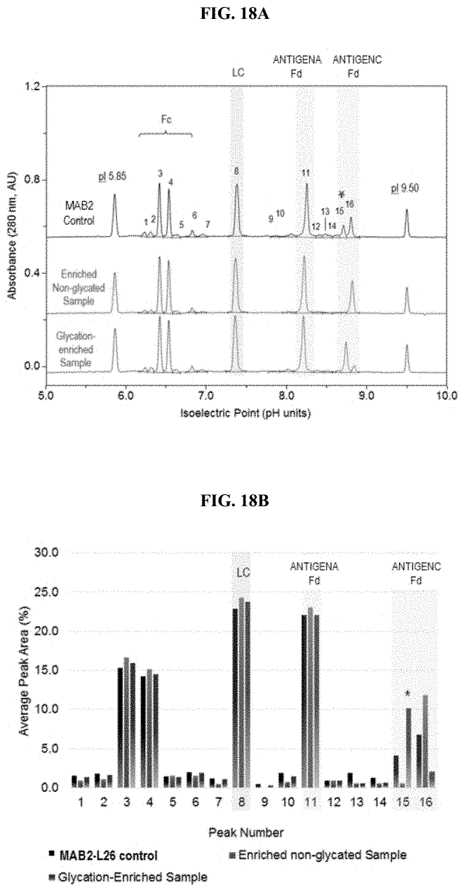

The enriched glycated species and non-glycated species were subjected to IdeS digestion and analyzed using DiCE under reduced/denatured conditions. As shown in FIG. 18A, the relative abundance of peak 15 (*) was higher in the glycation-enriched sample and was lower in the enriched non-glycated sample in comparing to peak 16. The average peak area in percentages corresponding to peak numbers are shown in FIG. 18B.

In comparing peaks 15 and 16 as shown in Table 2, the non-glycated sample is enriched for peak 16. However, the glycation-enriched sample is enriched for peak 15. The relative abundance of peak 15 (*) was higher in the glycation-enriched sample, while peak 16 was increased in the non-glycated sample. Based on the observation of these two major ANTIGENC Fd′ peaks, the quantification of glycation levels could be estimated by taking the relative ratio of peak 15 to the sum of peaks 15 and 16.

TABLE 2
Relative abundance of ANTIGENC
Fd’ peaks analyzed using DiCE.

| | |
|---|---|
| Peak 15 | Peak 16 |

| | | | | |
|---|---|---|---|---|
| Control MAB2-L26 | 4.1 | (0.2) | 6.8 | (0.3) |
| Non-glycated sample | 0.6 | (0.1) | 11.9 | (0.1) |
| Glycation-enriched | 10.2 | (0.1) | 2.1 | (0.1) |
| sample | | | | |

The quantification of the levels of glycation in MAB2 samples were further verified using peptide mapping as shown in Table 3. Table 3 shows the comparisons using CEX, peptide mapping and DiCE. The results indicated that the quantification using DiCE was comparable with peptide mapping.

TABLE 3
Quantification of glycation levels in MAB2 samples

| | % Glycation, | % Glycation, | % Glycatioon, |
|---|---|---|---|
| | CEX | Peptide Mapping | DiCE |
| Control MAB2-L26 | 31 | 38 | 37.7 |
| Non-glycated sample | <5 | 2 | 4.8 |
| Glycation-enriched | >95 | 84 | 82.9 |
| sample | | | |

### Example 7. Cell line Development In Monitoring Charge Variants

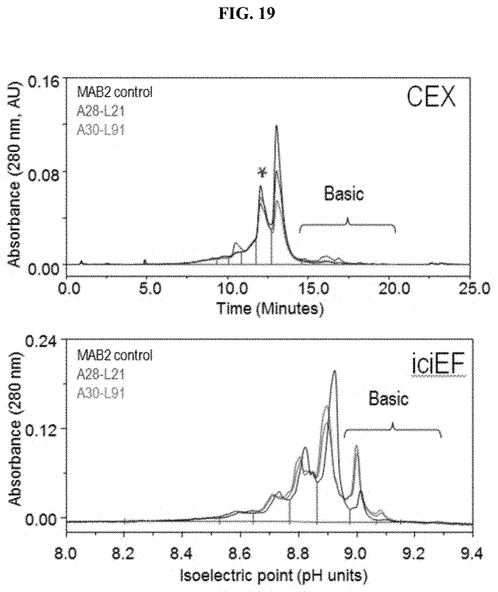

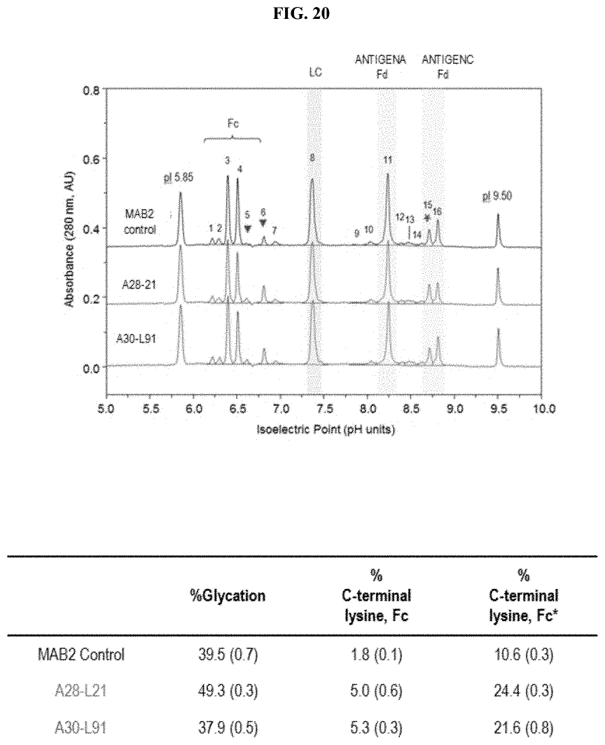

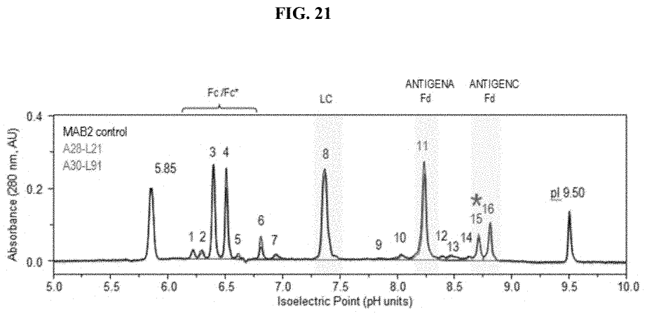

Cell lines were developed for producing antibodies by monitoring the charge variants of the produced antibodies. The cell line development was monitored using charge-based assays as a proxy for various post-translational modifications (PTMs) that may result from the new cell lines. Two cell lines, e.g. A28-L21 and A30-L91, were analyzed using CEX and icIEF against C1 clinical comparator sample to assess comparability. A discrepancy was observed between CEX and icIEF with respect to the total abundance of charge basic variants as shown in FIG. 19. A slight increase in glycation was observed in A28-L21 compared to the control sample and A30-L91. Both C2 cell lines showed an increase in C-terminal lysine predominately on the Fc*. When DiCE was used for MAB2 C2 cell selection, the samples reveal a mild increase in Fc C-terminal lysine (peak 5), but a significant increase in Fc* C-terminal lysine (peak 6) as shown in FIG. 20 and FIG. 21. DiCE analysis suggested that the increase in basic species observed in icIEF is due to unprocessed C-terminal lysine on the Fc domain, and the Fc* domain is less susceptible to clipping. In addition, the level of glycation for the A28-L21 clone was slightly higher than is typically observed for MAB2. DiCE can be used to rapidly assess levels of glycation on ANTIGENC to support cell line and process development of MAB2.

### Example 8. Monitoring Post-Translation Modification (PTM)

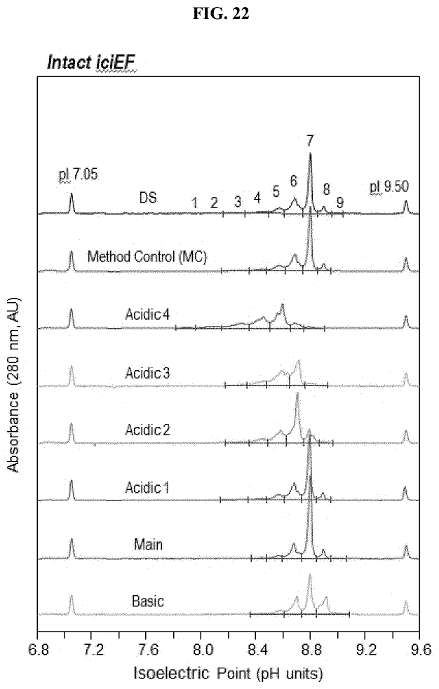

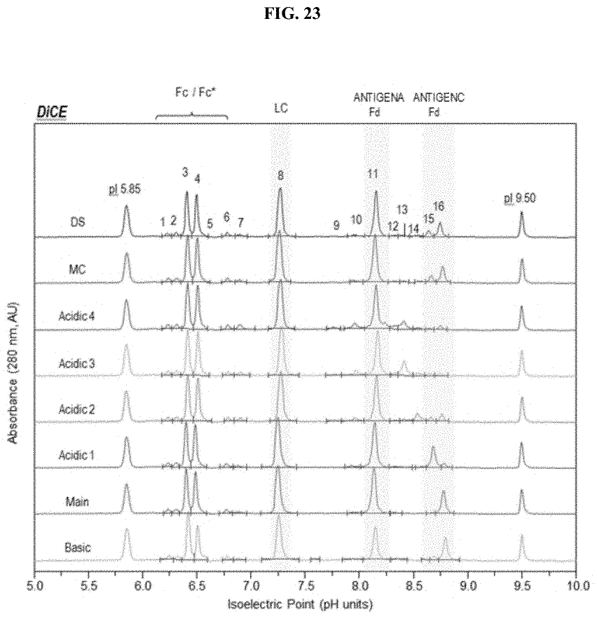

The intact icIEF and DiCE profiles for charge variants enriched by dual pH-salt gradient cation exchange chromatography revealed orthogonality between the two methods. While peaks corresponding to the Fc/Fc*, LC and ANTIGENA-Fd′ fragment remained relatively constant across all samples, the ANTIGENC Fd′ peaks (12-16) exhibited significant changes in distribution across the acidic fractions as shown in FIGS. 22 and 23. DiCE analysis of enriched charge variants of MAB2 suggested that the ANTIGENC arm may be more susceptible to PTMs compared to the ANTIGENA arm.

### Example 9. Analysis of Enriched Charge Variants of MAB5 Using DiCE

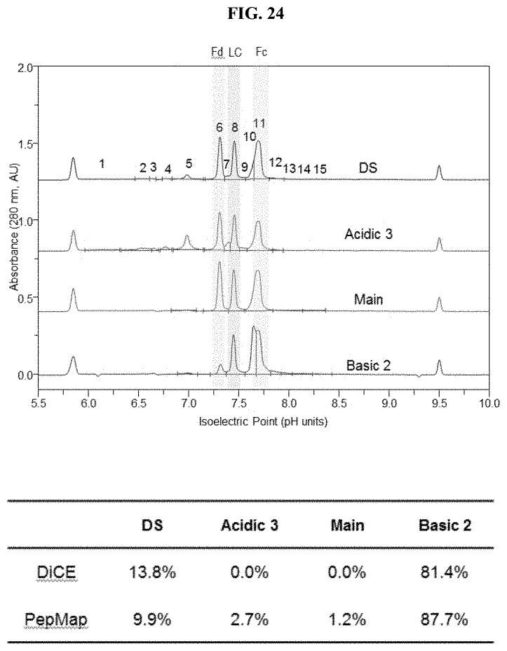

DiCE was used to analyze the enriched charge variants of MABS as shown in FIG. 24. Peaks 6, 8, and 11 represented the predominate charged species. Peak 6 corresponds to Fc. Peak 8 corresponds to LC. Peak 11 corresponds to the predominant Fd. Acidic 3 is specifically enriched for peaks 5, 7. Peak 10 has the highest abundance in enriched Basic 2 and likely corresponds to the MABS Fc fragment with unprocessed C-terminal lysine.

### Example 10. Analysis of Combo-Eb Using DiCE

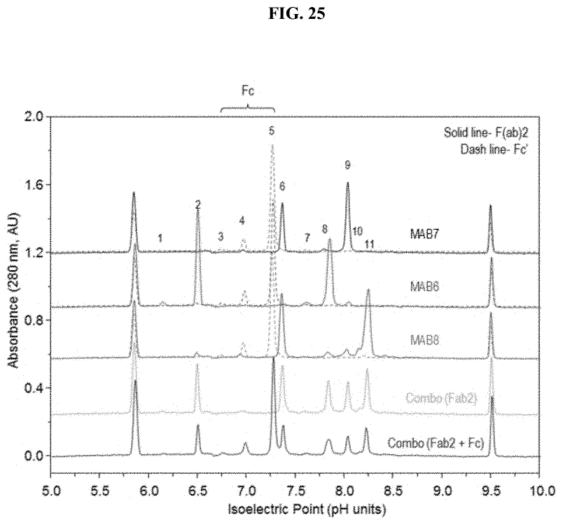

As shown in FIG. 25, peak 1-2 correspond to MAB6 LC. Peaks 3-5 correspond to the Fc/2 and present in all samples. Peak 6 represents the LC of MAB7 and MAB8. Their sequences are almost identical to each other. Peaks 7-8 correspond to MAB6 Fd fragment. Peak 9 represents to MAB7 Fd fragment. Peaks 10-11 correspond to MAB8 Fd fragment.

What is claimed is:

1. A method for identifying one or more variants of at least one peptide or protein, comprising:
treating said at least one peptide or protein with one or more digestion enzymes to generate two or more components of said at least one peptide or protein;
reducing or denaturing said components;
separating said two or more components of said at least one peptide or protein, wherein a separation profile is generated; and
identifying said variants, wherein identification is optionally based on said separation profile.

2. A method of claim 1, wherein said components are separated based on biophysical parameters.

3. The method of claim 1, wherein said digestion enzyme is an immunoglobulin G-degrading enzyme of Streptococcus pyogenes, sialidase, cysteine protease, endopeptidase, papain, endoproteinase Lys-C, pepsin, trypsin, carboxypeptidase B, protease, sialidase, exoglycosidase or a combination thereof.

4. The method of claim 1, further comprising treating said one or more components with one or more digestion enzymes in multiple phases to generate additional components.

5. The method of claim 1, wherein said reducing or denaturing conditions include use of urea, guanidinium chloride, dithiothreitol (DTT), Tris(2-carboxyethyl)phosphine hydrochloride (TCEP), organic solvents, alkaline solution, acid solution or a combination thereof.

6. The method of claim 2, wherein said components of said at least one peptide or protein are separated based on charge variants of said components.

7. The method of claim 1, wherein said components of said at least one peptide or protein are separated using isoelectric focusing electrophoresis method, a capillary isoelectric focusing electrophoresis method, an imaged capillary isoelectric focusing electrophoresis method, a chromatography coupled capillary electrophoresis method, capillary electrophoresis, a chromatography coupled imaged capillary electrophoresis method, a cation-exchange chromatography method or a liquid chromatography-mass spectrometry method.

8. The method of claim 1, further comprising quantifying or identifying said separated components of the at least one peptide or protein.

9. The method of claim 1, further comprising identifying the components of said at least one peptide or protein based on a comparison of a separation profile for at least one peptide or protein with a different charge variant.

10. The method of claim 9, further comprising quantifying the level of said variants.

11. The method of claim 10, wherein said variants of said at least one peptide or protein include variants from post-translation modification.

12. The method of claim 10, wherein said variants from glycation or C-terminal lysine of said components.

13. The method of claim 1, wherein said at least one peptide or protein is a drug, an antibody, a bispecific antibody, a monoclonal antibody, a fusion protein, an antibody-drug conjugate, an antibody fragment or a protein pharmaceutical product.

14. A system for identifying charge variants of at least one peptide or protein, comprising:
at least one peptide or protein;
a first digestion enzyme capable of generating components of said at least one peptide or protein;
an environment capable of reducing or denaturing said components of said at least one peptide or protein; and
an apparatus capable of separating said components of said at least one peptide or protein that have been reduced or denatured .

15. The system of claim 14, wherein the environment further comprises a second digestion enzyme capable of treating said components of said at least one peptide or protein.

16. The system of claim 15, wherein said first or second digestion enzyme is an immunoglobulin-degrading enzyme of Streptococcus pyogenes, sialidase, cysteine protease, endopeptidase, papain, endoproteinase Lys-C, pepsin, trypsin, carboxypeptidase B, or protease.

17. The system of claim 14, wherein said environment further comprises a reducing or denaturing agent, wherein said reducing or denaturing agent is urea, guanidinium chloride, reducing agents, dithiothreitol (DTT), Tris(2-carboxyethyl) phosphine hydrochloride (TCEP), organic solvents, alkaline solution, acid solution or a combination thereof.

18. The system of claim 14, wherein said apparatus separates based on charge heterogeneity.

19. The system of claim 14, wherein said apparatus is an isoelectric focusing apparatus, capillary isoelectric focusing electrophoresis apparatus, an imaged capillary isoelectric focusing electrophoresis apparatus, a chromatograph coupled capillary electrophoresis apparatus, a chromatograph coupled imaged capillary electrophoresis apparatus, a cation-exchange chromatograph apparatus, or a liquid chromatography-mass spectrometry apparatus.

20. The system of claim 14, wherein said at least one peptide or protein is a bispecific antibody.

21. The system of claim 14, wherein said at least one peptide or protein is a drug, an antibody, a bispecific antibody, a monoclonal antibody, a fusion protein, an antibody-drug conjugate, an antibody fragment or a protein pharmaceutical product.
